(19) United States
(12) Patent Application Publication
(10) Pub. No.: US 2003/0204193 A1
(43) Pub. Date: Oct. 30, 2003

(54) Suture anchor insertion tool

(76) Inventors: Stefan Gabriel, Mattapoisett, MA (US); Dennis Colleran, North Attleborough, MA (US); Paul Vincuilla, North Attleborough, MA (US); Paul Salvas, Norton, MA (US); Laird L. Hatch, Cave Creek, AZ (US); Paul O'Connor, Norfolk, MA (US)

Correspondence Address: JOEL R. PETROW, ESQ., Smith & Nephew, Inc., 1450 Brooks Road, Memphis, TN, 38116, US

(21) Appl. No.: 10/132,355

(22) Filed: Apr. 25, 2002

(51) Int. Cl. A61F 2/00

(52) U.S. Cl. 606/139; 606/104; 606/232

(57) ABSTRACT

A tool includes a housing adapted to receive an anchor with attached suture, and a suture retainer adapted to receive the attached suture. The suture retainer is configured to regulate deployment of the suture from the retainer such that deployment of the suture from the housing does not occur until a load applied to the suture corresponds to at least a selected minimum fixation strength of the anchor in bone. A method includes inserting a suture anchor at an attachment site, the suture anchor having an attached suture, and deploying the attached suture from a suture retainer when a load applied to the suture corresponds to at least a selected minimum fixation strength of the anchor in bone.

### BACKGROUND

Soft tissue, such as ligaments and tendons, after they have torn away from bone, can be reattached using suture. The surgeon inserts a suture anchor with an attached suture into the bone and ties the suture about the soft tissue to secure the soft tissue to the bone. The suture anchor is deployed within the bone in a manner that resists pull-out from the bone in response to forces exerted during healing that tend to draw the reattached ligament or tendon, and thus the suture and suture anchor, away from the bone.

### SUMMARY

According to one aspect of the invention, a tool includes a housing adapted to receive an anchor with attached suture, and a suture retainer coupled to the housing and adapted to receive the attached suture. The suture retainer is configured to regulate deployment of the suture from the housing such that deployment of the suture from the housing does not occur until a load applied to the suture corresponds to at least a selected minimum fixation strength of the anchor in bone.

Embodiments of this aspect of the invention may include one or more of the following features.

The suture retainer includes a suture hold for housing the attached suture, and a hold regulator configured to regulate deployment of the suture from the suture hold. The suture hold is a spool that rotates to unwind suture during deployment, and the hold regulator is a ratcheting mechanism configured to regulate rotation of the spool. The spool includes tabs, and the ratchet mechanism is a flexible member having a tip configured to contact the tabs to exert a rotation limiting force on the tabs related to the selected minimum fixation strength. The flexible member is configured and arranged such that the selected miminum fixation strength is not overcome if the anchor dislodges from tissue, but the selected miminum fixation strength is overcome and the spool rotates to deploy the suture if the anchor is lodged in the tissue.

The suture hold includes a needle dock configured to receive a needle attached to the suture. The needle dock is defined by a cutout portion of the suture hold.

In an illustrated embodiment, the suture hold is defined by a channel for receiving suture, and the hold regulator is in the form of a deformable tube. The hold regulator includes a compressor for radially compressing the deformable tube against suture to create a frictional force against the suture related to the minimum fixation strength.

The housing includes a handle and attached shaft. The handle defines a chamber for receiving the suture retainer. The minimum fixation strength of the anchor in bone is greater than about 3 or 4 lbs.

According to another aspect of the invention, a tool includes a needle dock that is configured to retain a suture needle and to release the suture needle upon application of a predetermined tension to a suture connected to the suture needle, the predetermined tension being related to a selected minimum fixation strength of a suture anchor in bone.

According to another aspect of the invention, a tool includes means for receiving an anchor with attached suture, and means for receiving the attached suture to regulate deployment of the suture from the tool such that a load on the suture required to initiate deployment of the suture from the tool corresponds to a selected minimum fixation strength of the anchor in bone.

According to another aspect of the invention, a method includes inserting a suture anchor with an attached suture at an attachment site, and deploying the attached suture from a suture retainer when a load applied to the suture corresponds to at least a selected minimum fixation strength of the anchor in bone.

Embodiments of this aspect of the invention may include one or more of the following features. The minimum anchor fixation strength is greater than about 3 lbs. The method includes housing the attached suture in a suture hold and regulating deployment of the suture from the suture hold. Regulating deployment of the suture from the suture hold includes regulating rotation of the suture hold. Regulating rotation of the suture hold includes flexing a flexible member contacting the suture hold. Regulating deployment of the suture from the suture hold includes generating friction between the suture and the suture hold. Generating friction includes pressing a member against the suture.

Advantages of the invention may include that the fixation of the suture anchor to bone is automatically tested under a predetermined load while the suture anchor is still loaded in the insertion tool. Thus, if there is inadequate fixation, the surgeon can further secure the anchor or secure the anchor in an alternate site without the need for reloading the suture or using a new tool. In addition, the needles are stored with their tips covered for safety and are automatically deployed from the storage position after insertion of the suture anchor. These advantages save a surgeon time, reduce the risk of injury to a surgeon, and improve the reproducibility of suture anchor insertion procedures.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a top view of a suture anchor insertion tool;

FIG. 1B is a side view of the insertion tool of FIG. 1A;

FIG. 2A is an exploded view of the insertion tool of FIG. 1A;

FIG. 2B is a sectional view of the insertion tool along lines 2B-2B of FIG. 1A;

FIG. 3A is an enlarged view of a spool and a spool chamber of the insertion tool of FIG. 1A;

FIG. 3B is a n enlarged back view of a portion of the spool of the insertion tool of FIG. 1A;

FIG. 3C is an enlarged side view of the spool of the insertion tool of FIG. 1A;

FIGS. 4A-4D are diagrammatic illustrations of the insertion tool of FIG. 1A shown at various stages during deployment of a suture anchor;

FIGS. 5A-5C are illustrations of the spool within the spool chamber shown at various stages during deployment of the suture anchor;

FIG. 6 is a graph of Force Resisting Rotation as a function of suture payout;

FIG. 7 is a perspective view of an alternative embodiment of a suture anchor insertion tool;

FIG. 8 is an exploded view of the insertion tool of FIG. 7;

FIG. 9A is a perspective view of the insertion tool of FIG. 7 shown partially assembled;

FIG. 9B shows one of the halves of a handle of the insertion tool of FIG. 7;

FIG. 10A is a top view of a n elongate member of the insertion tool of FIG. 7;

FIGS. 10B-10E are cross-sectional views of the elongate member taken along lines 10B-10B through 10D-10E, respectively, of FIG. 10A;

FIG. 11 is a perspective view of a needle retainer of the insertion tool of FIG. 7; and

FIGS. 12A-12D are diagrammatic illustrations of the insertion tool of FIG. 7 shown at various stages during deployment of a suture anchor.

### DETAILED DESCRIPTION

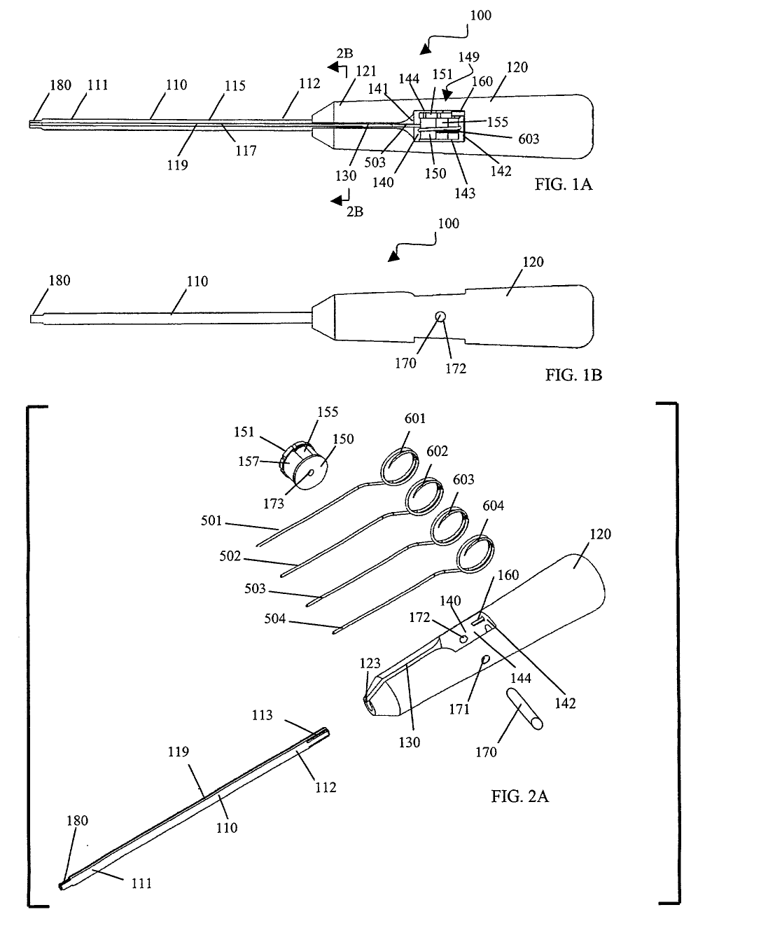

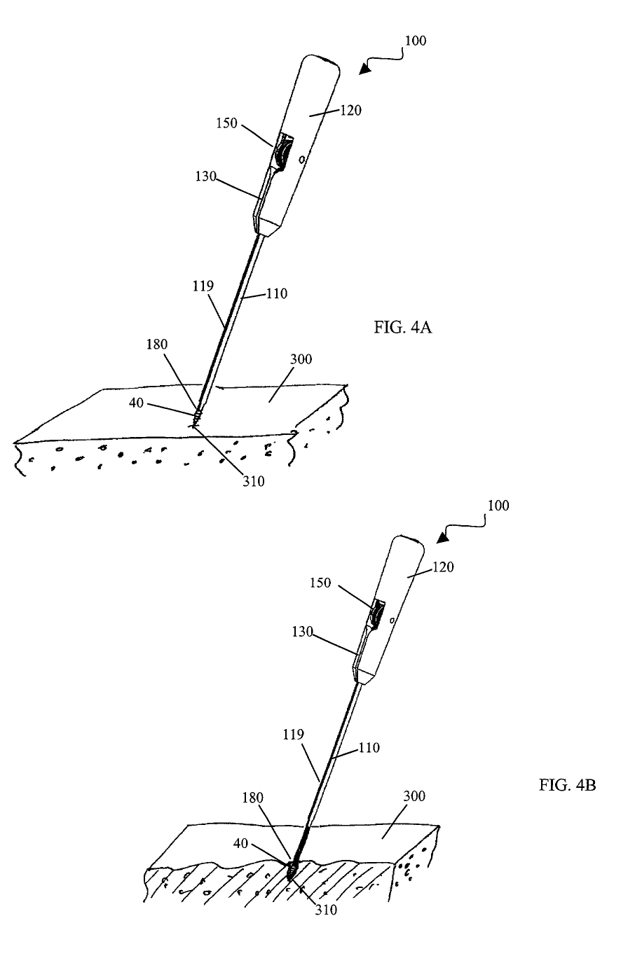

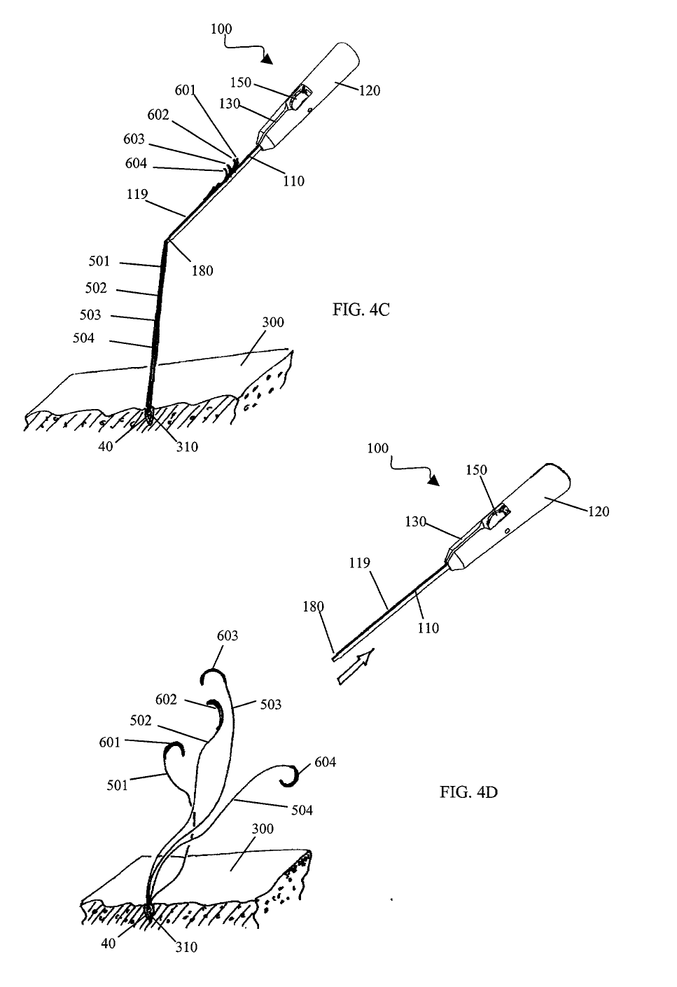

Referring to FIGS. 1A and 1B, a suture anchor insertion tool 100 includes a shaft 110 for housing a suture anchor 40 (FIG. 4A) with attached suture 503, and a handle 120 with a suture retainer 149 for regulating deployment of the suture from the tool. Shaft 110 has a distal portion 111 with a suture anchor receiver 180 for receiving the suture anchor, and a wall 115 defining a lumen 117 sized to contain at least one strand of a suture 503 extending from the suture anchor to handle 120. Handle 120 defines a channel 130 for receiving suture 503 and a chamber 140 in which suture retainer 149 is located. Suture retainer 149 includes a suture hold, e.g., a rotatable spool 150, around which suture 503 is wound, and a hold regulator 160, e.g., a ratchet mechanism, that interacts with spool 150 to control unwinding of suture 503 from spool 150 and deployment of suture 503 from insertion tool 100 such that the load required to initiate deployment of the suture is related to a minimum retention force lodging the anchor in bone, as discussed further below.

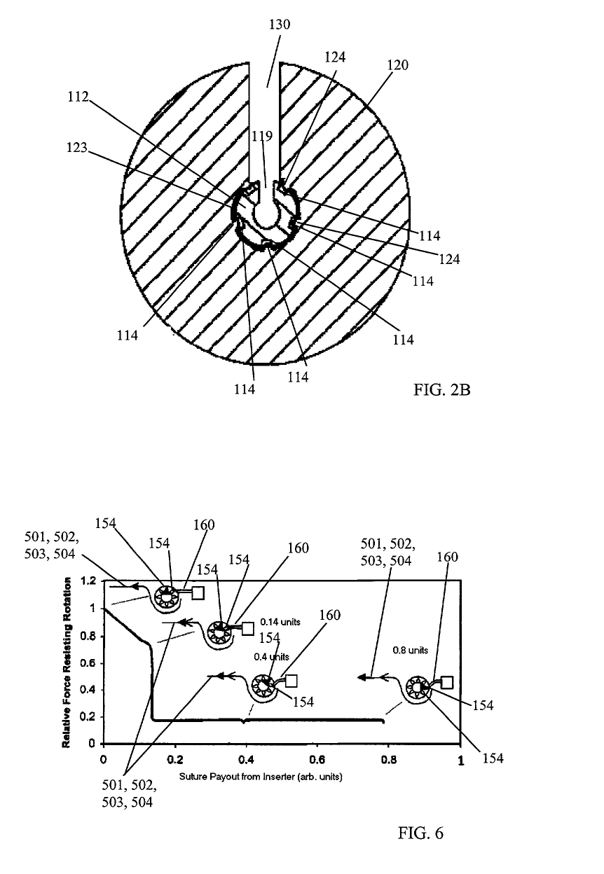

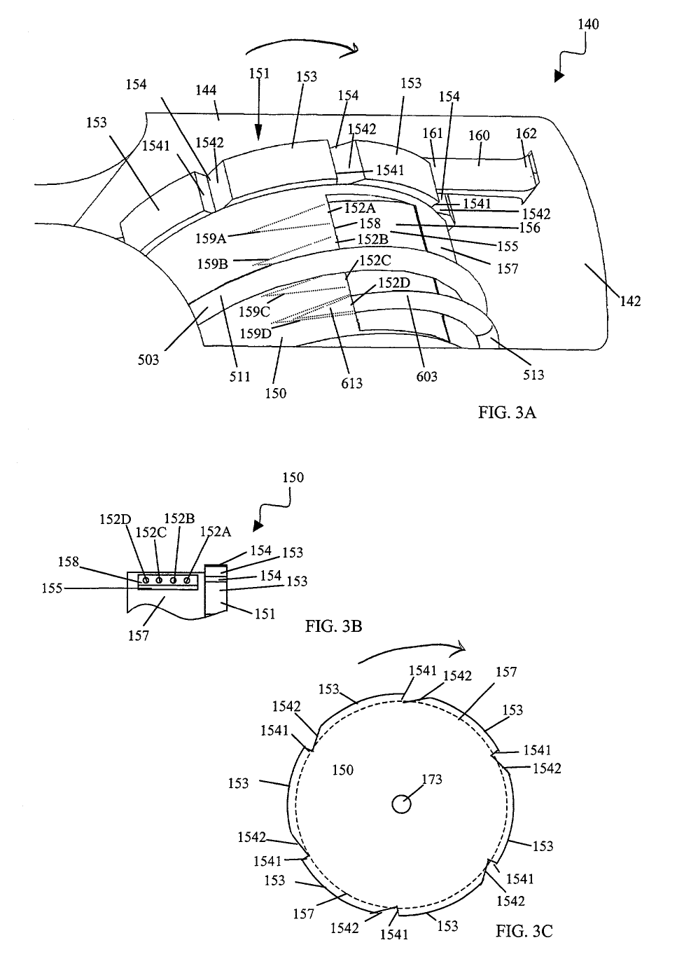

For the sake of clarity of illustration, only a single suture 503 is shown extending into spool chamber 140 in FIGS. 1A and 3A. However, as shown in FIG. 2A, four sutures 501, 502, 503, and 504 extend into spool chamber 140 and interact with spool 150 in the manner described below with respect to suture 503. Referring also to FIG. 2A, lumen 117 and channel 130 contain sutures 501, 502, 503, and 504 extending from a retained suture anchor (not shown) proximally into chamber 140.

Chamber 140 is bounded by side walls 143, 144, a distal wall 141, and a proximal wall 142 of handle 120. Side walls 143, 144 define holes 171, 172 that receive a compression fit dowel 170. Dowel 170 rotatably mounts spool 150 through a hole 173 in spool 150. Spool 150 is generally cylindrical in shape and includes a circumferential suture winding surface 157 and a raised, notched lip 151. Sutures 501, 502, 503, 504 are wound about winding surface 157. Notched lip 151 is contacted by spool regulator 160 which extends distally from proximal wall 142 for regulating unwinding of the sutures 501, 502, 503, 504 from the winding surface 157, as discussed further below. Spool 150 also defines a cutout 155. Cutout 155 houses portions of one or more arcuate needles 601, 602, 603, 604 at the end of sutures 501, 502, 503, 504.

Referring to FIGS. 3A and 3B, cutout 155 is bounded by a base face 156 and a wall 158, and includes arcuate conical chambers 159A, 159B, 159C, and 159D. Base face 156 extends radially inward from winding surface 157 to meet wall 158. Wall 158 defines holes 152A, 152B, 152C, and 152D that open into arcuate conical chambers 159A, 159B, 159C, and 159D. Suture 503 has an end 513 in loop 511 joined to arcuate needle 603. Needle 603 itself terminates in a tip 613. Suture 503 extends proximally from channel 130 into spool chamber 140 and winds in a counterclockwise direction (as viewed in FIG. 3A) around spool 150 to form a loop 511. Needle 603 extends from suture end 513 on winding surface 157 into cutout 155. Needle 603 enters hole 152D in cutout 155 and tip 613 is received in conical chamber 159D by a compression fit so that a force of about zero to two pounds is required to remove needle 603. For example, the radius of curvature of arcuate conical chambers 159A, 159B, 159C, and 159D can be selected to be slightly larger than the radius of curvature of arcuate needles 601, 602, 603, and 604, resulting in a releasable compression fit therebetween. The removal force can be applied by tension upon suture 503 to automatically withdraw needle 603 from conical chamber 159D. The retention force holding the needles in the conical chambers is preferably less than the minimum fixation strength for deploying the suture.

Referring also to FIG. 3C, to regulate deployment of suture 503 from the spool 150, raised lip 151 includes an alternating series of tabs 153, e.g., six tabs, and notches 154, e.g., six notches, around the circumference of spool 150. Notches 154 are each bounded by a respective radial face 1541 and an obtuse face 1542. Spool regulator 160 rests in a notch 154 and exerts a force on spool 150 that resists clockwise rotation of spool 150 when a load is applied to the wound suture. If enough tension is placed on the suture, i.e., tension at least equal to the minimum fixation strength, the force exerted on spool 150 by regulator 160 is overcome, allowing the spool to rotate and the suture to be deployed.

As shown in FIG. 3A, spool regulator 160 is tooth-shaped and has a distal end 161 and a proximal end 162. Proximal end 162 increases in thickness approaching proximal wall 142 while distal end 161 decreases in thickness approaching spool 150. The thickness of spool regulator 160 at various positions can be selected to provide a desired stiffness for regulating unwinding of suture 503 from spool 150 and deployment of suture 503 from insertion tool 100. Deployment of suture 503 from spool 150 results when suture 503 is pulled distally with sufficient force to rotate spool 150 in the clockwise direction. With spool 150 in a zero payout storage position, i.e., with suture 503 wound tightly enough about spool 150 to retain a suture anchor on suture anchor receiver 180 as illustrated in FIG. 3, spool regulator 160 extends distally from proximal wall 142 into a first notch 154 and contacts faces 1541, 1542.

Referring to FIG. 2B, tube 110 has a proximal end 112 terminating in a grooved portion 113 having a series of longitudinally extending depressions 114. Grooved portion 113 is received in a complementary grooved chamber 123 at the distal end of the handle. Grooved chamber 123 includes a series of longitudinally extending ridges 124 that mate with depressions 114 to prevent rotation between the tube and handle.

Referring to FIG. 4A, an anchor 40 received in anchor receiver 180 of insertion tool 100 is retained in insertion tool 100 by tension along sutures 501, 502, 503, and 504. The illustrated anchor 40 is designed to be inserted into a tissue, e.g., bone 300, by rotation. Anchor receiver 180 is designed to transmit a torque to anchor 40 and is formed by, e.g., a hex head, a Phillips head, or a flat head that complements a mounting portion (not shown) on anchor 40. Other tissue anchors, e.g., anchors inserted by compressive loads, can be used with insertion tool 100.

Still referring to FIG. 4A, in use, an operator advances insertion tool 100 preloaded with anchor 40 and sutures 501, 502, 503, and 504 and needles 601, 602, 603, and 604, to the surgical site against bone 300. The operator then positions insertion tool 100 to orient anchor 40 toward a selected position 310 in bone 300.

Referring to FIG. 4B, the operator then drives anchor 40 into position 310 by applying a torque to handle 120. The torque is transmitted along shaft 110 to anchor 40.

During the positioning and driving illustrated in FIGS. 4A and 4B, spool 150 remains in the zero payout storage position of FIG. 3A. After anchor 40 has been driven a sufficient depth into position 310, the operator withdraws insertion tool 100 away from bone 300. If the fixation of anchor 40 to bone 300 is sufficiently strong, sutures 501, 502, 503, and 504 and needles 601, 602, 603, and 604 will payout from spool 150 by rotating spool 150 in the clockwise direction, as discussed further below.

Referring to FIG. 4C, continued withdrawal of insertion tool 100 away from bone 300 draws needles 601, 602, 603, and 604 out of conical chambers 159A, 159B, 159C, and 159D and into slot 119. This is done automatically, without further intervention by the operator. The arcuate shape of needles 601, 602, 603, and 604 is accommodated by slot 119 in conjunction with lumen 117.

Referring to FIG. 4D, continued withdrawal of insertion tool 100 away from bone 300 draws needles 601, 602, 603, and 604 through receiver 180 and releases needles 601, 602, 603, and 604 and sutures 501, 502, 503, and 504 from tool 100.

The rotation of spool 150 and thus the release of suture from tool 100 is regulated by spool regulator 160. The withdrawal of insertion tool 100 away from bone 300 causes the unwinding of sutures 501, 502, 503, and 504 from winding surface 157 and the extraction of needles 601, 602, 603, and 604 from conical chambers 159A, 159B, 159C, and 159D. In particular, before the extraction of needles 601, 602, 603, and 604, the withdrawal of insertion tool 100 away from bone 300 causes tension in sutures 501, 502, 503, and 504 which apply a clockwise torque to spool 150. The ability of this clockwise torque to rotate spool 150 is regulated by spool regulator 160.

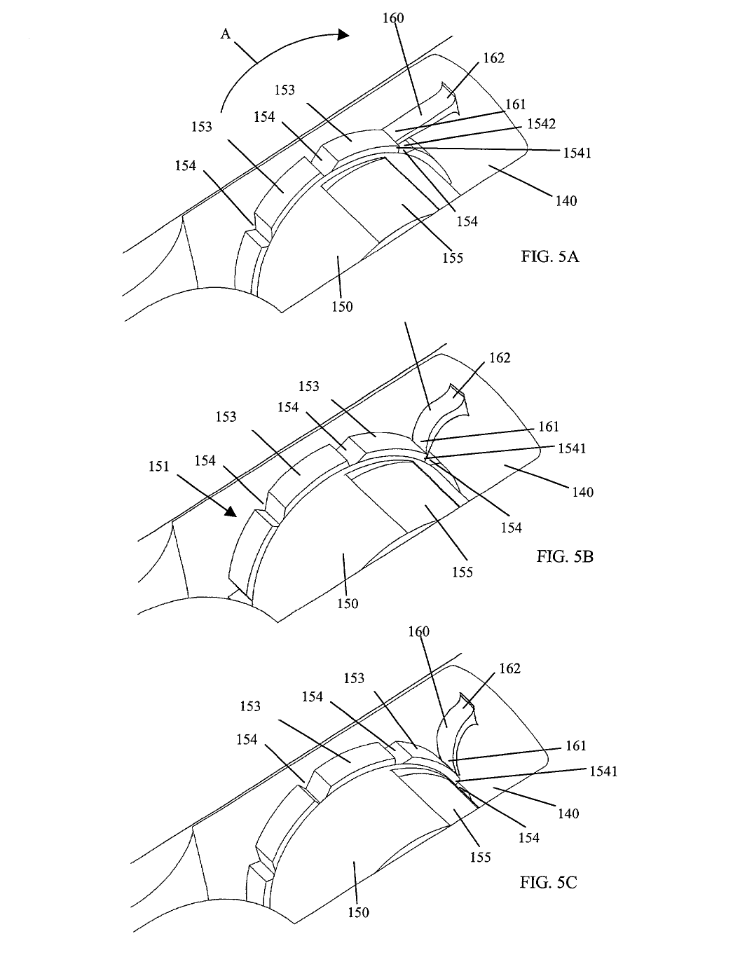

Referring to FIGS. 5A-5C, in which sutures 501, 502, 503, 504 and needles 601, 602, 603, 604 are omitted for clarity, with spool 150 in the zero payout storage position of FIG. 5A, the clockwise torque on spool 150 causes spool 150 to rotate clockwise, arrow A, such that distal end 161 engages face 1541. The radial position of the zero payout storage position can be selected such that the instantaneous force applied by face 1541 to spool regulator 160 is primarily compressive with relatively little flexure. Moreover, spool regulator 160 can be tuned to have a relatively high compressive stiffness and a relatively low flexural stiffness by, e.g., adjusting the geometry (e.g., thickness) of distal end 161 and proximal end 162 of spool regulator 160.

Referring to FIG. 5B, further clockwise rotation of spool 150 away from the zero payout storage position decreases the relative magnitude of the compressive component of the force applied by face 1541 to spool regulator 160 while increasing the relative magnitude of the flexural component of the instantaneous force applied by face 1541 to spool regulator 160. This results in increased flexion of spool regulator 160.

Referring to FIG. 5C, further clockwise rotation of spool 150 moves distal end 161 of regulator 160 out of notch 154 and distal end 161 begins to slide along tab 153.

Referring also to FIG. 6, as a result of the positioning and structure of regulator 160 and spool 150, the Force Resisting Rotation of spool 150, i.e., the selected minimum fixation strength which corresponds to the force needed to overcome the resistance of regulator 160, is relatively high when spool 150 is in the zero payout storage position of FIG. 3. This allows an operator to test the fixation of anchor 40 in position 310. In particular, by requiring a relatively large force before suture deployment (i.e., while anchor 40 is still loaded in insertion tool 100), an operator can test if anchor 40 pulls away from position 310 automatically under a predetermined load, without unloading anchor 40. Suitable test loads are, for example, greater than about 3, 4 or 5 pounds, 10 pounds or 15 pounds, depending on the type of anchor and the type of bone into which the anchor is placed, e.g., for lower quality bones, lower test loads are appropriate.

The rotation of spool 150 away from the zero payout storage position results in deployment of sutures 501, 502, 503, and 504, and a decrease in the Force Resisting Rotation. This is illustrated in the region between zero payout and about 0.127 units payout in the graph. The rate of decrease in the Force Resisting Rotation and the payout over which this decrease occurs can be controlled, e.g., by adjusting the position of spool regulator 160 on proximal wall 142, the geometry and materials of distal end 161 and proximal end 162 of spool regulator 160, and the diameter of spool 150.

The movement of spool regulator 160 out of notch 154A results in a sudden drop in the Force Resisting Rotation, illustrated at about 0.127 units payout in the graph. Indeed, the Force Resisting Rotation drops to a relatively static level of about 20% of the Force Resisting Rotation in the zero payout storage position. This static Force Resisting Rotation arises primarily due to friction between distal end 161 of spool regulator 160 and tab 153A, and can also be tuned as needed.

The Force Resisting Rotation remains substantially at the static level during further rotation of spool 150 and deplotment of sutures 501, 502, 503, and 504. Exceptions occur when subsequent notches 154 pass beneath spool regulator 160, illustrated at about 0.4 and 0.8 units payout in the graph. In particular, the Force Resisting Rotation drops slightly as distal end 161 of spool regulator 160 slides radially inward along faces 1542. However, as spool 150 rotates further, distal end 161 moves out of notches 154 and slides along subsequent tabs 153 again experiencing friction causing the static level Force Resisting Rotation.

Spool regulator 160 thus regulates the retention of sutures 501, 502, 503, and 504 on spool 150 by varying the Force Resisting Rotation of spool 150 with the deployment of sutures 501, 502, 503, and 504.

Other embodiments are within the scope of the following claims.

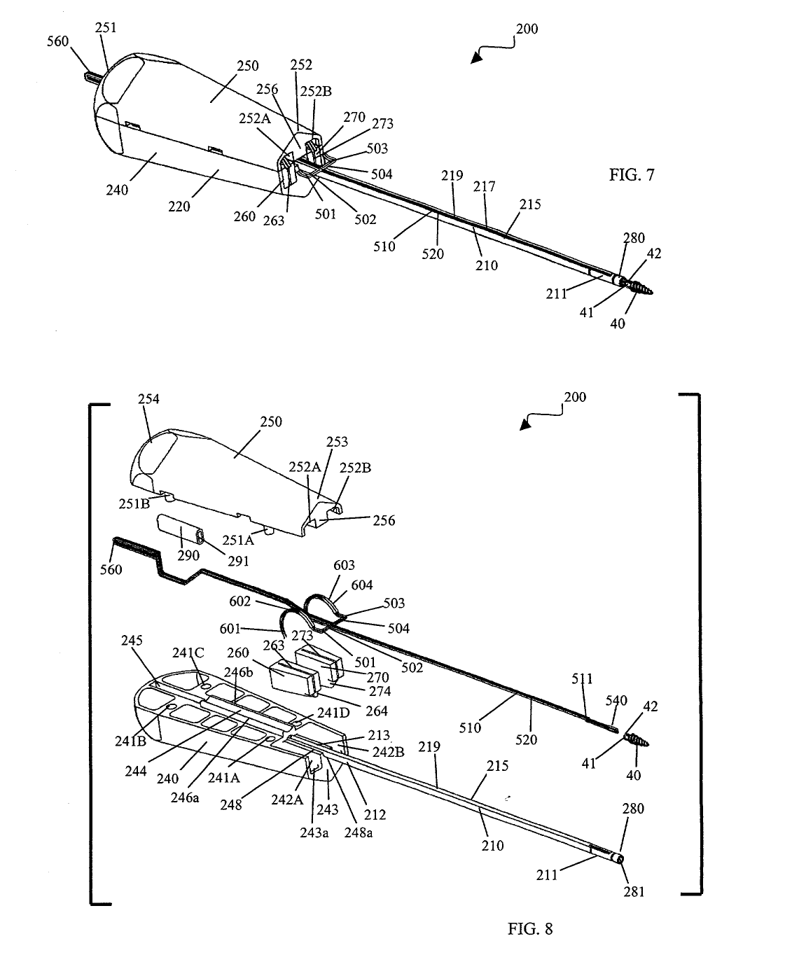

For example, referring to FIG. 7, a suture anchor insertion tool 200 for housing a suture anchor with attached suture and regulating deployment of the suture includes a handle 220 joined to an elongate member 210 terminating at distal portion 211 in a tissue anchor receiver 280. Member 210 has a wall 215 defining a lumen 217 with a slot 219 extending through wall 215. Lumen 217 is sized to contain at least two sutures 510, 520 routed as described below.

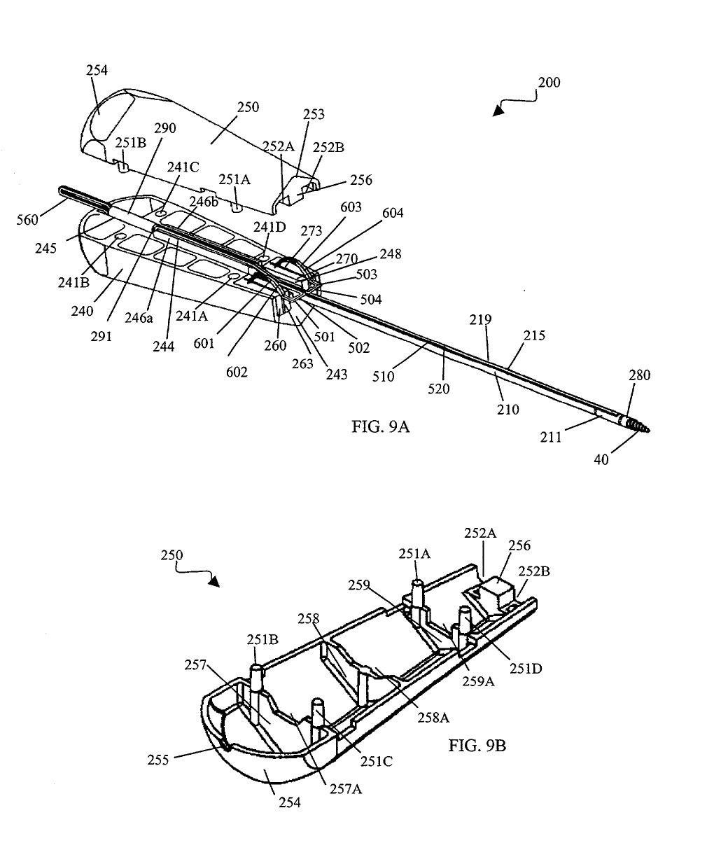

Handle 220 is formed of two complementary handle halves 240, 250. Sutures 510, 520 pass through an anchor 40, through elongate member 210, and between handle halves 240, 250. Referring also to FIGS. 8 and 9A, handle 220 includes a tubular suture restrictor 290 that regulates deployment of the sutures 510, 520 from tool 200 such that the load required to initiate deployment of sutures 510, 520 is related to a minimum retention force lodging anchor 40 in bone, as discussed further below. Suture 510 has ends 501, 502 each terminating in a suture needle 601, 602, respectively, and suture 520 has ends 503, 504 each terminating in a suture needle 603, 604, respectively. Handle 220 includes needle retainers 260, 270 for housing sutures needles 601, 602, 603, 604.

Sutures 510, 520 are routed through tool 200 with suture 510 extending proximally from needle 601 through handle 220, then folding back distally to anchor 40, through anchor 40 and proximally back to needle 602. Likewise, suture 520 extends proximally from needle 603 through handle 220, then folds back distally to anchor 40, through anchor 40 and proximally back to needle 604.

Referring also to FIGS. 9A and 9B, handle half 250 has a distal end 253 defining a pair of cutouts 252A, 252B separated by a ridge 256. Handle half 240 has a distal end 243 defining a pair of corresponding cutouts 242A, 242B separated by an elongate member receptacle 248. When the handle halves are assembled, each pair of cutouts 252A and 242A, 252B and 242B forms a chamber housing a respective needle retainer 260, 270.

The interior of handle half 250 has four nubs 251A, 251B, 251C, 251D, and the interior of handle half 240 defines a series of opening 241A, 241B, 241C, 241D for receiving nubs 251A, 251B, 251C, 251D, respectively, in a compression fit to couple handle halves 240, 250.

Elongate member receptacle 248 defined in handle half 240 has an opening 248a at distal end 243 of handle half 240 through which a proximal end 212 of elongate member 210 extends into receptacle 248. End 212 can be sealed within receptacle 248 using, e.g., epoxy. Proximal of receptacle 248, handle half 240 has a pair of substantially parallel ridges 245, 246 defining a groove 244 therebetween for receiving suture 510, 520. The interior of handle half 240 has three lateral support ribs 257, 258, 259, each defining a cutout 257a, 258a, 259a, respectively. Cutouts 257A, 258A, 259A accommodate ridges 245, 246 when halves 240, 250 are fitted together and provide clearance for passage of the suture through handle 220. Suture passes from groove 244 to lumen 217 through slot 219 when end 212 is sealed in receptacle 248.

Proximal of groove 244, handle half 240 defines a chamber 245 that receives a tubular suture restrictor 290. Restrictor 290 is a deformable structure made from, for example, TYGON tubing. Suture restrictor 290 defines a channel 291 through which suture 510, 520 passes. Suture restrictor 290 is dimensioned to press against the suture in channel 291 when restrictor 290 is fit within chamber 245 to generate friction that regulates payout of suture 510, 520 from tool 200.

The interior of handle half 240 defines a hole 255 in proximal end 254. Hole 255 accommodates suture loops 560 formed when sutures 510, 520 fold back distally. Suture 510, 520 thus passes from needles 601, 603, respectively, through groove 244, through restrictor channel 291, through hole 255 forming loops 560, back through hole 255, through cannel 291 and groove 244, through slot 219 into lumen 217, through a hole 41 in anchor 40, back through lumen 217 and slot 219 to needles 602, 604, respectively.

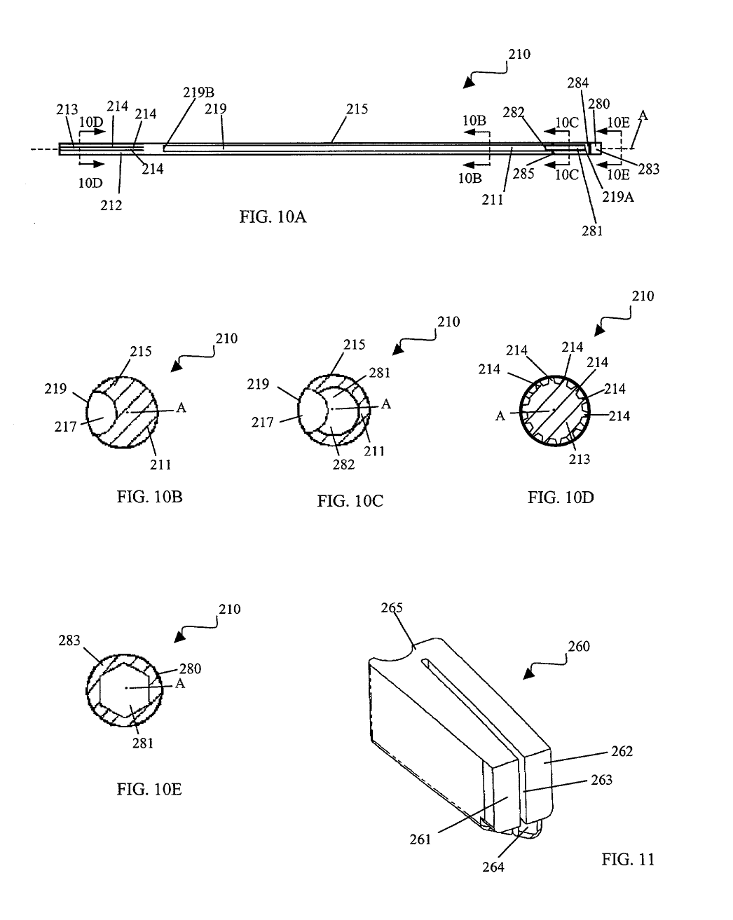

Referring to FIGS. 10A-10E, proximal end 212 of elongate member 210 includes a knurled region 213. Member 210 is attached to handle 220 by, e.g., molding the handle onto the shaft, with knurled region 213 acting to limit any possible rotation of member 210 relative to handle 220. Knurled region 213 include, e.g., 10-20 longitudinal ridges 214 protruding radially outward.

Tissue anchor receiver 280 includes a pair of positioning lines 284, 285 spaced about 10 mm apart. Line 284 indicates to the user the insertion depth of the anchor in bone, and line 285 is a reference mark for aiding in determining the tissue thickness above the bone. Lumen 217 and slot 219 extend between a distal wall 219A and a proximal wall 219B. Tissue anchor receiver 280 defines a cavity 281 extending distally from a wall 282 of member 210. Suture passes from lumen 217 into cavity 281. Cavity 281 has a solid-walled distal region 283 extending distally from wall 219A. Cavity 281 in distal region 283 is hex-shaped for mating with a male hexagonal mounting portion 42 (FIG. 8) of anchor 40 so that an insertion torque can be transmitted from tool 200 to anchor 40. The enlarged cavity 281 allows passage of the suture needles through distal region 283.

Insertion tool 200 can be machined or molded from, e.g., biocompatible metals or plastics.

Referring to FIGS. 8 and 11, each needle retainer 260, 270 defines a respective slot 263, 273 and includes a respective lip 264, 274. Slots 263, 273 receive and retain sutures needles 601 and 602, and 603 and 604, respectively. Needle retainers 260, 270 are deformable structures made from, for example, silicone, and are deformed slightly when receiving sutures needles 601, 602, 603, 604 to form a compression fit that is releasable when tension is applied to sutures 510, 520. Needle retainers 260, 270 are maintained in cutouts 242A and 242B by contact of lips 264, 274 against ledges 243a.

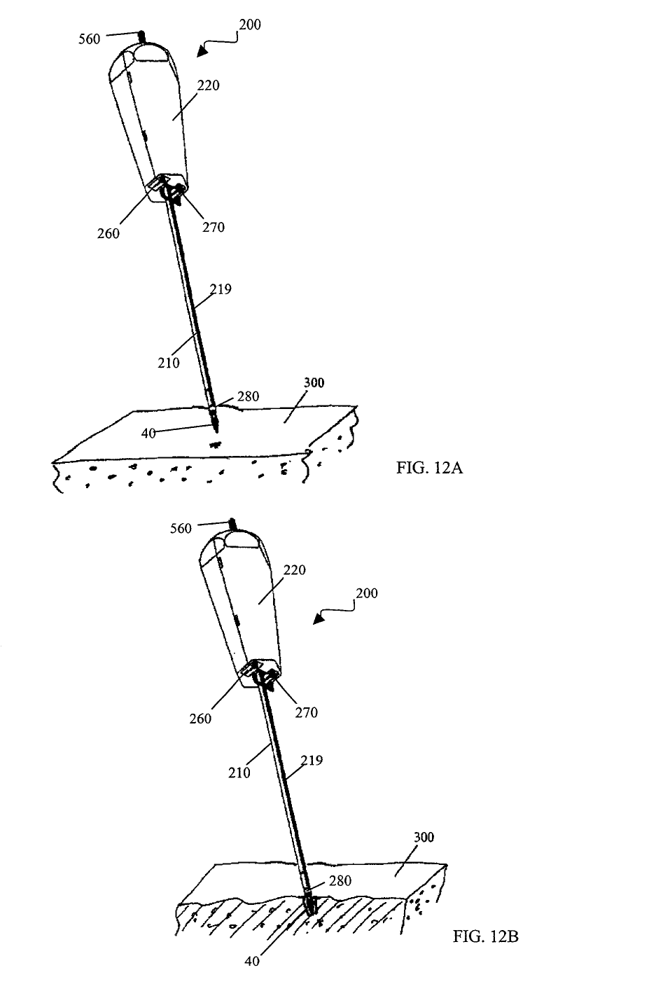

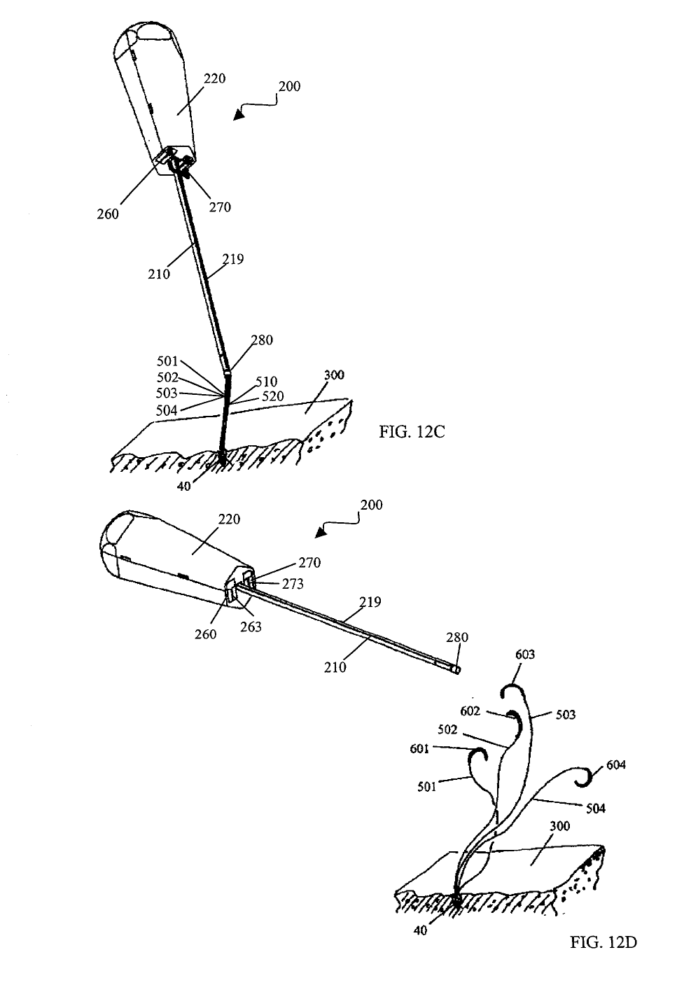

Referring to FIG. 12A, in use, an operator positions insertion tool 200 loaded with anchor 40 and associated sutures 510, 520 and needles 601, 602, 603, and 604 against tissue, e.g., bone 300. Referring to FIG. 12B, the operator then drives anchor 40 into bone 300 by applying a torque to handle 220. The torque is transmitted along elongate member 210 to receiver 280 and anchor 40. Depending upon the type of anchor, anchor 40 can be driven using, e.g., compressive loads, and anchor 40 can be driven into a pre-drilled hole in the bone.

Referring to FIG. 12C, after anchor 40 has been driven a sufficient depth into bone, the operator withdraws tool 200 proximally from bone 300. If the fixation of anchor 40 to bone 300 is sufficiently strong, sutures 510, 520 are drawn from tool 200. Restrictor 290 regulates deployment of sutures 510, 520 from tool 200 by generating friction between tool 200 and sutures 510, 520. The amount of friction generated can be varied, e.g., by changing the material of restrictor 290 or the diameter of channel 291. By manipulating these or other factors, the load on the sutures 510, 520 required to initiate deployment of the sutures from the retainer can correspond to a minimum anchor fixation strength. For example, the load on the sutures 510, 520 required to initiate deployment is in excess of 3 lbs, and preferably in excess of 4 lbs.

During withdrawal of sutures 510, 520 from tool 200, the length of loop 360 progressively shortens until loop 560 is drawn into handle 250. Access to loop 560 allows an operator to retighten sutures 510, 520 by pulling on the sutures if the sutures accidentally loosen during positioning. The operator can hold the suture that is initially drawn out of the distal end of tool 200 during continued withdrawal of the insertion tool to limit sliding of the suture relative to the anchor.

Referring to FIG. 12D, continued withdrawal of insertion tool 200 draws sutures 510, 520 out of handle 250 and applies a tensile force to suture needles 601-604, pulling them out of slots 263, 273 in needle retainers 260, 270. This occurs automatically, without further intervention by the operator. The friction on needles 601-604 is selected such that the needles are retained until a sufficient force corresponding to the selected minimum fixation strength is applied to sutures 510, 520. The friction on the needles can be varied, e.g., by changing the width of slots 263, 273 or the materials of needle retainers 260, 270. The operator individually manipulates each needle through cavity 281 in distal region 283 of the member 210 (FIG. 10). Once needles 601-604 are released from tool 200, the operator can use sutures 510, 520 to fasten tissue, e.g., soft tissue, to bone 300.

Further embodiments are within the scope of the following claims. For example, insertion tool 100 may be assembled by a surgeon or other operator prior to use. The operator positions anchor 40 on receiver 180 and runs sutures 501, 502, 503, and 504 up lumen 117 into channel 130 and spool chamber 140 behind and around spool 150. The operator then inserts needles 601, 602, 603, and 604 into conical chambers 159A, 159B, 159C, and 159D and advances spool 150 in the counter-clockwise direction. The counter-clockwise advancement of spool 150 draws distal end 161 of regulator 160 along tabs 153 and obtuse faces 1542. Moreover, the operator draws sutures 501, 502, 503, and 504 around winding surface 157, forming a series of loops such as loop 511 of FIG. 3. The operator continues to advance spool 150 in the counter-clockwise direction until a sufficient tension is maintained in sutures 501, 502, 503, and 504 to retain anchor 40 is retained on receiver 180.

Regulation of suture deployment may be accomplished in other ways. Referring particularly to insertion tool 200, one or more deformable elements can be placed anywhere along the path formed over member receptacle 248 and through the channel formed using groove 244 and restrictor 290 traversing handle 220. In particular, a foam pad can be fixed to the base of ridge 256 to compress suture 510, 520 between handle halves 240, 250.

What is claimed is:

1. A tool comprising:
a housing adapted to receive an anchor with attached suture; and
a suture retainer coupled to the housing and adapted to receive the attached suture, the suture retainer configured to regulate deployment of the suture from the housing such that deployment of the suture from the housing does not occur until a load applied to the suture corresponds to at least a selected minimum fixation strength of the anchor in bone.

2. The tool of claim 1 wherein the suture retainer is configured to apply a retention force to the suture, the retention force being overcome when the imposed on the suture corresponds to the minimum fixation strength.

3. The tool of claim 1 wherein the suture retainer comprises a suture hold for receiving the attached suture, and a hold regulator configured to regulate deployment of the suture from the suture hold.

4. The tool of claim 3 wherein the suture hold comprises a spool that rotates to unwind suture during deployment, and the hold regulator comprises a ratcheting mechanism configured to regulate rotation of the spool.

5. The tool of claim 4 wherein the spool includes tabs.

6. The tool of claim 5 wherein the ratchet mechanism comprises a flexible member having a tip configured to contact the tabs to exert a rotation limiting force on the tabs related to the selected minimum fixation strength.

7. The tool of claim 6 wherein the flexible member is configured and arranged such that the selected miminum fixation strength is not overcome if the anchor dislodges from tissue, but the selected miminum fixation strength is overcome and the spool rotates to deploy suture if the anchor is lodged in the tissue.

8. The tool of claim 3 wherein the suture hold includes a needle dock configured to receive a needle attached to the suture.

9. The tool of claim 8 wherein the needle dock is defined by a cutout portion of the suture hold.

10. The tool of claim 3 wherein the suture hold comprises a channel for receiving suture.

11. The tool of claim 3 wherein the hold regulator comprises a deformable tube.

12. The tool of claim 11 wherein the hold regulator comprises a compressor for radially compressing the deformable tube against suture to create a frictional force against the suture related to the minimum fixation strength.

13. The tool of claim 1 wherein the housing includes a handle and attached shaft.

14. The tool of claim 13 wherein the handle defines a chamber for receiving the suture retainer.

15. The tool of claim 1 wherein the minimum anchor fixation strength is greater than about 3 lbs.

16. The tool of claim 15 wherein the minimum anchor fixation strength is greater than about 4 lbs.

17. A tool comprising:
a needle dock configured to retain a suture needle and to release the suture needle upon an application of predetermined tension to a suture connected to the suture needle, the predetermined tension being related to a selected minimum fixation strength of a suture anchor in bone.

18. A tool comprising:
means for receiving an anchor with attached suture; and
means for receiving the attached suture to regulate deployment of the suture from the tool such that a load on the suture required to initiate deployment of the suture from the tool corresponds to a selected minimum fixation strength of the anchor in bone.

19. A method comprising:
inserting a suture anchor at an attachment site, the suture anchor having an attached suture; and
deploying the attached suture from a suture retainer when a load applied to the suture corresponds to at least a selected minimum fixation strength of the anchor in bone.

20. The method of claim 19 wherein the minimum fixation strength is greater than about 3 lbs.

21. The method of claim 19 further comprising receiving the attached suture in a suture hold, and regulating deployment of the suture from the suture hold.

22. The method of claim 21 wherein regulating deployment of the suture from the suture hold comprises regulating rotation of the suture hold.

23. The method of claim 22 wherein regulating rotation of the suture hold comprises flexing a flexible member contacting the suture hold.

24. The method of claim 21 wherein regulating deployment of the suture from the suture hold comprises generating friction between the suture and the suture hold.

25. The method of claim 24 wherein generating friction comprises pressing a member against the suture.
